(19) United States
(12) Patent Application Publication
(10) Pub. No.: US 2023/0284938 A1
(43) Pub. Date: Sep. 14, 2023

(54) INFORMATION PROCESSING APPARATUS AND INFORMATION PROCESSING PROGRAM

(71) Applicant: FUJIFILM CORPORATION, Tokyo (JP)

(72) Inventors: Haruyasu NAKATSUGAWA, Kanagawa (JP); Tomohide HIRAGAMI, Kanagawa (JP); Nobuya KITAMURA, Kanagawa (JP); Yasuhisa KANEKO, Kanagawa (JP); Kenji NAGAMIYA, Kanagawa (JP); Yasuyuki HOSONO, Kanagawa (JP)

(21) Appl. No.: 18/318,735

(22) Filed: May 17, 2023

Related U.S. Application Data
(63) Continuation of application No. PCT/JP2021/043020, filed on Nov. 24, 2021.

(30) Foreign Application Priority Data
Nov. 30, 2020 (JP) 2020-199170

(51) Int. Cl. A61B 5/145

(52) U.S. Cl. CPC A61B 5/14532

(57) ABSTRACT

An information processing apparatus comprising at least one processor, wherein the at least one processor is configured to: acquire a measured value, which is measured in a sample test, together with time information indicating a measurement time point of the measured value; acquire a monitoring value which is obtained by monitoring biological information having a correlation with the measured value; and associate the measured value with the monitoring value at the measurement time point indicated by the time information.

### CROSS REFERENCE TO RELATED APPLICATIONS

This application is a continuation of International Application No. PCT/JP2021/043020, filed on Nov. 24, 2021, which claims priority from Japanese Patent Application No. 2020-199170, filed on Nov. 30, 2020. The entire disclosure of each of the above applications is incorporated herein by reference.

### BACKGROUND

### Technical Field

The present disclosure relates to an information processing apparatus and an information processing program.

### Related Art

In the related art, there has been disclosed a technique of monitoring biological information of a user who wears a wearable terminal such as a smart watch and using the monitored biological information for health diagnosis, disease prevention, health promotion, and the like. For example, JP2019-052696A describes a technique of performing diagnosis on the basis of vital data (for example, pulse rate, blood pressure, body temperature, and respiratory rate) of an examinee acquired by a vital band worn on the arm of the examinee, and test data (for example, a result of blood test). Further, for example, JP2019-072467A describes a technique of measuring biological information (for example, blood glucose) of a user by a biological information sensor attached to the test target site of the user, and correcting the biological information on the basis of the food and drink information of the user (for example, the ingested food and the intake amount, and the intake time).

Further, various forms of a measurement device for measuring a level of blood glucose of a user such as a diabetic patient are known. For example, as a level of blood glucose measurement device, there is known a measurement device (hereinafter, referred to as “self-blood glucose measurement device”) that measures a level of blood glucose by attaching a blood obtained by puncturing one's fingertip to a sensor. Although the self-blood glucose measurement device is able to measure the level of blood glucose more accurately, the burden on the user is increased due to the pain at the time of puncturing, the running cost, and the like. As a result, it is difficult to measure fluctuation in the level of blood glucose over time. Meanwhile, there is also known a measurement device (hereinafter, referred to as “continuous blood glucose measurement device”) that measures not the level of blood glucose itself but a level of glucose of an interstitial fluid having a correlation with the level of blood glucose by a sensor mounted on the skin of a user. Since the continuous blood glucose measurement device measures the level of glucose of the interstitial fluid, the accuracy is inferior to that of the self-blood glucose measurement device, but it is possible to measure fluctuation in the level of blood glucose with time. For example, JP2019-018005A describes a technique of continuously measuring a level of glucose in an interstitial fluid by using a continuous blood glucose measurement device and presenting an appropriate timing for causing a user to measure a level of blood glucose by using a self-blood glucose measurement device on the basis of the level of glucose.

Meanwhile, in a case where measurement is performed by a sample test at a specific time point, for example, as in the case of measurement by the above-mentioned self-blood glucose measurement device, the measurement result may be better or worse than usual. For example, the level of blood glucose may fluctuate in accordance with a situation such as after waking up, before going to bed, before meals, after meals, at rest, during exercise, even during a single day. Further, the level of blood glucose may fluctuate as compared with other days in accordance with the physical condition, the amount of activity, the sleeping time, the content of the meal, and the like on that day.

Even in a case where the measurement results that are better or worse than usual are used for health examination, disease prevention, health promotion, and the like, it is considered that a correct determination cannot be made. Therefore, in recent years, there has been a demand for a technique capable of acquiring an appropriate measurement result in consideration of the above-mentioned fluctuations.

### SUMMARY

The present disclosure provides an information processing apparatus and an information processing program capable of acquiring the appropriate measurement result.

According to a first aspect of the present disclosure, there is provided an information processing apparatus comprising at least one processor, in which the processor is configured to: acquire a measured value, which is measured in a sample test, together with time information indicating a measurement time point of the measured value; acquire a monitoring value which is obtained by monitoring biological information having a correlation with the measured value; and associate the measured value with the monitoring value at the measurement time point indicated by the time information.

According to a second aspect of the present disclosure, in the above-mentioned aspect, the processor may be configured to associate the acquired measured value with the monitoring value at the measurement time point indicated by the time information, after the measured value and the time information are acquired.

According to a third aspect of the present disclosure, in the above-mentioned aspect, the processor may be configured to: acquire the monitoring value at each of a plurality of time points; and associate the measured value with a plurality of the monitoring values in a predetermined period, which includes at least one of a time point before or a time point after the measurement time point indicated by the time information, among the monitoring values at the plurality of time points.

According to a fourth aspect of the present disclosure, in the third aspect, the processor may be configured to: derive a tendency of fluctuation in the monitoring value in a predetermined period, which includes at least one of a time point before or a time point after the measurement time point indicated by the time information, on the basis of the plurality of the monitoring values associated with the measured value; and estimate a tendency of fluctuation in the measured value in the period, which includes at least one of a time point before or a time point after the measurement time point indicated by the time information, from the tendency of fluctuation in the monitoring value on the basis of correlation data in which a correlation between the measured value and the monitoring value is predetermined.

According to a fifth aspect of the present disclosure, in the fourth aspect, the measured value may be a level of blood glucose, the monitoring value may be at least one of a level of glucose included in interstitial fluid, sweat, or saliva, an electrocardiographic signal, a blood pressure, or a body temperature which has a correlation with the level of blood glucose, and the processor may be configured to: acquire timing information indicating whether the measurement time point is fasting or postprandial; and estimate the tendency of fluctuation in the measured value in the period, which includes at least one of a time point before or a time point after the measurement time point indicated by the time information, from the tendency of fluctuation in the monitoring value, on the basis of the correlation data corresponding to timing indicated by the timing information, in the correlation data corresponding to fasting and postprandial, respectively.

In the sixth aspect of the present disclosure, in the fourth or fifth aspect, the measured value may be a level of blood glucose, the monitoring value may be at least one of a level of glucose included in interstitial fluid, sweat, or saliva, an electrocardiographic signal, a blood pressure, or a body temperature which has a correlation with the level of blood glucose, and the processor may be configured to: acquire meal information indicating a content of a meal that a person from whom the measured value is obtained eats before the measurement of the measured value; and estimate the tendency of fluctuation in the measured value in the period, which includes at least one of a time point before or a time point after the measurement time point indicated by the time information, from the tendency of fluctuation in the monitoring value, on the basis of the correlation data corresponding to the content of the meal indicated by the meal information, in a plurality of pieces of the correlation data different for each content of the meal.

According to a seventh aspect of the present disclosure, in the fourth to sixth aspects, the processor may be configured to derive a range of fluctuation in the monitoring value in a predetermined period, which includes at least one of a time point before or a time point after the measurement time point indicated by the time information, as the tendency of fluctuation in the monitoring value.

According to an eighth aspect of the present disclosure, in the fourth to seventh aspects, the processor may be configured to output a comment corresponding to the estimated tendency of fluctuation in the measured value.

According to a ninth aspect of the present disclosure, in the fourth to eighth aspects, the processor may be configured to perform first evaluation on the measured value on the basis of the estimated tendency of fluctuation in the measured value.

According to a tenth aspect of the present disclosure, in the ninth aspect, the processor may be configured to output a comment corresponding to the first evaluation.

According to an eleventh aspect of the present disclosure, in the third to tenth aspects, the processor may be configured to: derive a tendency of fluctuation in the monitoring value in a predetermined period, which includes at least one of a time point before or a time point after the measurement time point indicated by the time information, on the basis of the plurality of the monitoring values associated with the measured value; and perform first evaluation on the measured value on the basis of the tendency of fluctuation in the monitoring value.

According to a twelfth aspect of the present disclosure, in the above-mentioned aspect, the processor may be configured to perform second evaluation on the measured value on the basis of a deviation between a reference value of the monitoring value at the measurement time point indicated by the time information and the monitoring value at the measurement time point indicated by the time information.

According to a thirteenth aspect of the present disclosure, in the twelfth aspect, the processor may be configured to output a comment corresponding to the second evaluation.

According to a fourteenth aspect of the present disclosure, in the above-mentioned aspect, the processor may be configured to perform third evaluation on the measurement time point indicated by the time information on the basis of the monitoring value associated with the measured value.

According to a fifteenth aspect of the present disclosure, in the fourteenth aspect, the processor may be configured to output a comment corresponding to the third evaluation.

According to a sixteenth aspect of the present disclosure, in the above-mentioned aspect, the processor may be configured to output the monitoring value associated with the measured value.

According to a seventeenth aspect of the present disclosure, there is provided an information processing apparatus comprising at least one processor, in which the processor may be configured to: acquire a monitoring value which is obtained by monitoring biological information having a correlation with a measured value which is measured in a sample test; and recommend measurement of the measured value in a case where a deviation between the monitoring value and a reference value of the monitoring value is smaller than a predetermined threshold value.

According to an eighteenth aspect of the present disclosure, in the above-mentioned aspect, the measured value may be a level of blood glucose, and the monitoring value may be at least one of a level of glucose included in interstitial fluid, sweat, or saliva, an electrocardiographic signal, a blood pressure, or a body temperature which has a correlation with the level of blood glucose.

According to a nineteenth aspect of the present disclosure, there is provided an information processing program for causing a computer to execute processing of: acquiring a measured value, which is measured in a sample test, together with time information indicating a measurement time point of the measured value; acquiring a monitoring value which is obtained by monitoring biological information having a correlation with the measured value; and associating the measured value with the monitoring value at the measurement time point indicated by the time information.

According to the above-mentioned aspect, the information processing apparatus and the information processing program of the present disclosure are able to acquire appropriate measurement results.

### BRIEF DESCRIPTION OF THE DRAWINGS

FIG. 1 is a schematic configuration diagram of an information processing system.

FIG. 2 is a diagram showing an example of a level of blood glucose.

FIG. 3 is a diagram showing an example of a level of glucose in an interstitial fluid.

FIG. 4 is a graph showing daily fluctuations of a level of glucose in the interstitial fluid.

FIG. 5 is a block diagram showing an example of a hardware configuration of the information processing apparatus.

FIG. 6 is a block diagram showing an example of a functional configuration of the information processing apparatus.

FIG. 7 is a diagram showing an example of correlation data between a level of blood glucose and a level of glucose in the interstitial fluid.

FIG. 8 is a diagram showing an example of correlation data between the level of blood glucose and the level of glucose in the interstitial fluid.

FIG. 9 is a diagram showing an example of a screen displayed on a display.

FIG. 10 is a diagram showing an example of the screen displayed on the display.

FIG. 11 is a flowchart showing an example of first evaluation processing.

FIG. 12 is a diagram showing an example of correlation data between a level of postprandial blood glucose and a level of glucose in the interstitial fluid.

FIG. 13 is a diagram showing an example of correlation data between a level of blood glucose and a level of glucose in the interstitial fluid for each content of the meal.

FIG. 14 is a diagram for explaining a reference value of the level of glucose in the interstitial fluid.

FIG. 15 is a flowchart showing an example of second evaluation processing.

FIG. 16 is a diagram showing a third evaluation.

FIG. 17 is a flowchart showing an example of third evaluation processing.

FIG. 18 is an example of a diagram which is output as a result of measurement recommendation processing.

FIG. 19 is a flowchart showing an example of the measurement recommendation processing.

### DETAILED DESCRIPTION

Examples of embodiments for carrying out the technique of the present disclosure will be hereinafter described in detail with reference to the drawings.

### FIRST EXEMPLARY EMBODIMENT

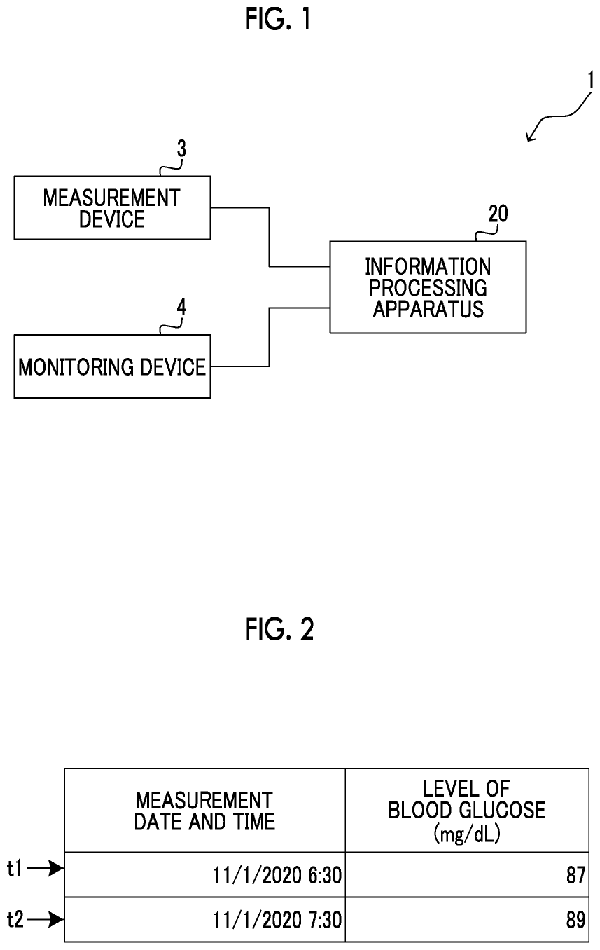

An example of a configuration of an information processing system 1 according to the present exemplary embodiment will be described with reference to FIG. 1. As shown in FIG. 1, the information processing system 1 includes an information processing apparatus 20, a measurement device 3, and a monitoring device 4. The information processing apparatus 20 and the measurement device 3 can be communicated with each other and the information processing apparatus 20 and the monitoring device 4 can be communicated with each other through wired or wireless communication (for example, Wi-Fi (registered trademark), Bluetooth (registered trademark), radio frequency identification (RFID), or the like).

The measurement device 3 is a device that performs a sample test. The measurement device 3 has a function of transmitting the measured value, which is measured in the sample test, to the information processing apparatus 20 together with the time information indicating the measurement time point t of the measured value. The measurement device 3 includes a non-volatile storage unit implemented by a storage medium such as a central processing unit (CPU), a hard disk drive (HDD), a solid state drive (SSD), and a flash memory, and a memory as a temporary storage region. Further, the measurement device 3 includes an input/output unit such as a mouse, a keyboard, a display, and a touch panel, and a network interface (I/F) that performs wired or wireless communication between the information processing apparatus 20 and an external network (not shown in the drawing).

The monitoring device 4 is a device that monitors biological information having a correlation with the measured value over time. The monitoring device 4 has a function of transmitting to the information processing apparatus 20 monitoring values at a plurality of time points which are obtained by monitoring the biological information over time. The “monitoring the biological information over time” means that the biological information is monitored at predetermined time intervals (for example, 15-minute intervals) without the user giving a monitoring instruction each time. It should be noted that the monitoring device 4 may monitor the biological information over time, and may also monitor the biological information even in a case where an instruction is issued by the user.

The monitoring device 4 includes a processor, a memory as a temporary storage region, a sensor, and a network I/F that performs wired or wireless communication between the information processing apparatus 20 and an external network (not shown in the drawing). The processors, memories, sensors, and network I/Fs may consist of an application specific integrated circuit (ASIC) for monitoring the biological information.

The present exemplary embodiment will describe an example of applying a device that measures a level of blood glucose by a blood test as the measurement device 3 and monitoring a level of glucose included in the interstitial fluid, which has a correlation with the level of blood glucose, over time as the monitoring device 4. The blood is an example of a sample, the level of blood glucose is an example of a measured value, and the level of glucose included in the interstitial fluid (hereinafter, referred to as “the level of glucose in the interstitial fluid”) is an example of the monitoring value.

As the measurement device 3 for measuring the level of blood glucose, for example, a portable self-blood glucose measurement device, in which a user punctures his/her fingertip to attach blood obtained to the sensor to measure the level of blood glucose, can be applied. Further, for example, it is possible to apply a stationary blood glucose measurement device that cause a medical worker such as a doctor, a nurse, or a laboratory technician to perform a blood test more precisely than a self-blood glucose measurement device using blood collected from a user and that is installed in a testing institution such as a hospital.

FIG. 2 shows an example of the levels of blood glucose measured by blood tests using the measurement device 3 for one user and measurement dates and times as an example of the time information. FIG. 2 shows that the level of blood glucose measured at the measurement time point t1 indicating “Nov. 1, 2020, 6:30” is 87 mg/dL and the level of blood glucose measured at the measurement time point t2 indicating “Nov. 1, 2020, 7:30” is 89 mg/dL. In such a case, the term “measurement dates and times” (that is, the measurement time points t of the measured values) indicates time points at which bloods are collected from a user. The term does not indicate time points, at which the blood tests performed by the measurement device 3 are completed, time points at which an acquisition unit 10 receives the level of blood glucose (details will be described later), and the like.

As the monitoring device 4 for monitoring the level of glucose in the interstitial fluid, for example, a device having a needle-like filament inserted subcutaneously of the user and measuring the level of glucose in the interstitial fluid by the filament can be applied (for example, refer to JP2016-520379A).

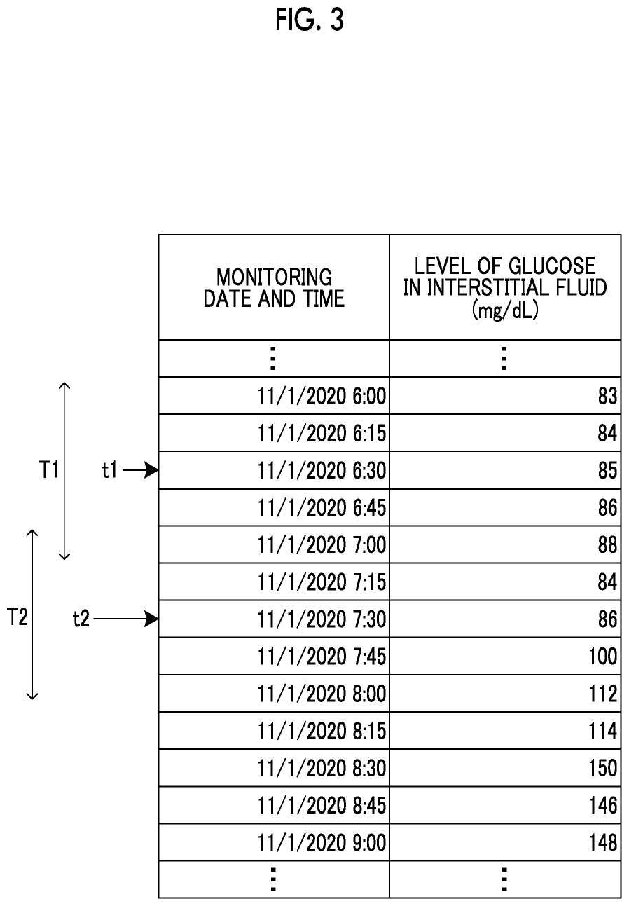

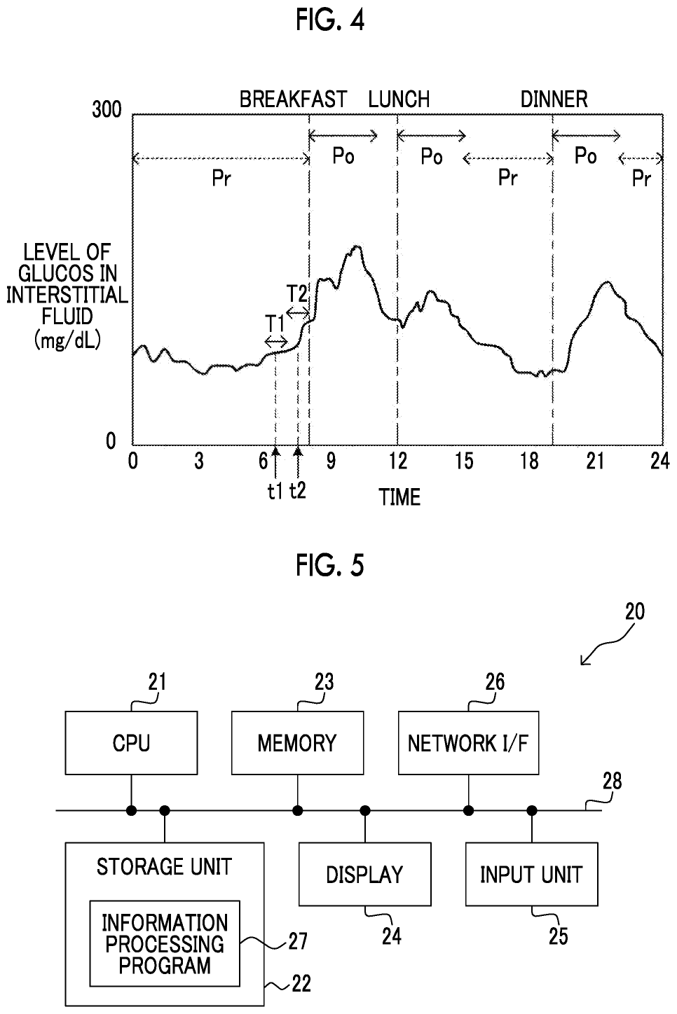

FIG. 3 shows an example of the levels of glucose in the interstitial fluid monitored by the monitoring device 4 at intervals of 15 minutes for a single user, and monitoring dates and times thereof. Further, FIG. 4 shows a graph of daily fluctuation in the levels of glucose in the interstitial fluid monitored by the monitoring device 4. FIG. 4 is a graph in which the horizontal axis represents the time and the vertical axis represents the level of glucose in the interstitial fluid. FIGS. 3 and 4 show the time points corresponding to the measurement time points t1 and t2 of the levels of blood glucose shown in FIG. 2. FIG. 4 shows the timing at which the user has breakfast, lunch, and dinner.

Meanwhile, as shown in FIG. 4, it is known that the level of blood glucose fluctuates in accordance with a situation such as before meals, after meals, at rest, during exercise, after waking up, and before going to bed, even during a single day. In a case where the levels of blood glucose are measured by the measurement device 3, the levels of blood glucose may be better or worse than usual in accordance with the measurement time points t. Even in a case where the levels of blood glucose that are better or worse than usual are used for health examination, disease prevention, health promotion, and the like, it is considered that a correct determination cannot be made.

Therefore, the information processing apparatus 20 according to the present exemplary embodiment evaluates whether the level of blood glucose is an appropriate value (whether it is a result in which the level of blood glucose is better or worse than usual) on the basis of the level of glucose in the interstitial fluid at the measurement time point t of the level of blood glucose, that is, whether the level of blood glucose is measured at an appropriate time point. Hereinafter, an example of a configuration of the information processing apparatus 20 according to the present exemplary embodiment will be described.

First, an example of a hardware configuration of the information processing apparatus 20 according to the present exemplary embodiment will be described with reference to FIG. 5. As shown in FIG. 5, the information processing apparatus 20 includes a CPU 21, a non-volatile storage unit 22, and a memory 23 as a temporary storage region. Further, the information processing apparatus 20 includes a network I/F 26 that performs wired or wireless communication with a display 24 such as a liquid crystal display, an input unit 25 such as a keyboard and a mouse, and the measurement device 3, the monitoring device 4, and an external network (not shown in the drawing). The CPU 21, the storage unit 22, the memory 23, the display 24, the input unit 25, and the network I/F 26 are connected to each other through a bus 28 such as a system bus and a control bus such that various types of information can be transmitted and received.

The storage unit 22 is implemented by, for example, a storage medium such as an HDD, an SSD, and a flash memory. The storage unit 22 stores the information processing program 27 according to the present exemplary embodiment. The CPU 21 reads out the information processing program 27 from the storage unit 22, expands the information processing program 27 into the memory 23, and executes the expanded information processing program 27. The CPU 21 is an example of the processor of the present disclosure. As the information processing apparatus 20, for example, it is possible to apply various computers such as a smartphone and a personal computer.

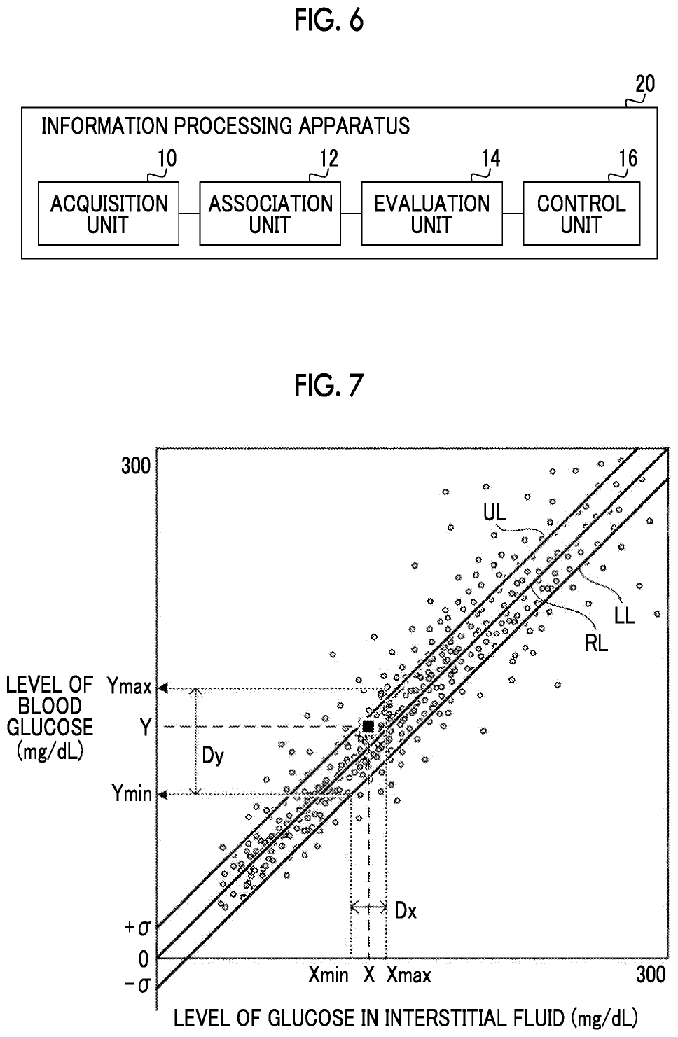

Next, an example of a functional configuration of the information processing apparatus 20 according to the present exemplary embodiment will be described with reference to FIG. 6. As shown in FIG. 6, the information processing apparatus 20 includes the acquisition unit 10, an association unit 12, an evaluation unit 14, and a control unit 16. The CPU 21 functions as the acquisition unit 10, the association unit 12, the evaluation unit 14, and the control unit 16 by executing the information processing program 27.

The acquisition unit 10 acquires the levels of blood glucose and the time information from the measurement device 3 (refer to FIG. 2). Further, the acquisition unit 10 acquires the levels of glucose in the interstitial fluid at a plurality of time points from the monitoring device 4 (refer to FIG. 3). Specifically, the acquisition unit 10 acquires the levels of glucose in the interstitial fluid from the monitoring device 4 at a plurality of time points including at least a time point corresponding to the measurement time point t of the level of blood glucose indicated by the time information.

The association unit 12 associates the level of blood glucose acquired by the acquisition unit 10 with the level of glucose in the interstitial fluid at the measurement time point t indicated by the time information. Specifically, the association unit 12 associates the levels of blood glucose with a plurality of levels of glucose in the interstitial fluid in a predetermined period T, which includes at least one of a time point before or a time point after the measurement time point t indicated by the time information, among the levels of glucose in the interstitial fluid at the plurality of time points acquired by the acquisition unit 10. The “period T” may be defined by, for example, a predetermined time (for example, 30 minutes), or may be defined by a time until the monitoring of the level of glucose in the interstitial fluid is performed for a predetermined number of times (for example, a time until monitoring is performed five times).

In the examples of FIGS. 2 and 3, the association unit 12 associates the level of blood glucose “87” at the measurement time point t1 indicated by the time information, with the five levels of glucose in the interstitial fluid “83”, “84”, “85”, “86”, and “88” in the period T1 including 30 minutes before and after the measurement time point t1. Similarly, the association unit 12 associates the level of blood glucose “89” at the measurement time point t2 indicated by the time information, with the five levels of glucose in the interstitial fluid “88”, “84”, “86”, “100”, and “112” in the period T2 including 30 minutes before and after the measurement time point t2.

That is, the association between the level of blood glucose and the level of glucose in the interstitial fluid performed by the association unit 12 is performed after the level of blood glucose and the time information are acquired. By associating the level of blood glucose with the level of glucose in the interstitial fluid at the measurement time point t at which the level of blood glucose is measured after the acquisition of the level of blood glucose, it is possible to evaluate the measured level of blood glucose, on the basis of the level of glucose in the interstitial fluid.

The evaluation unit 14 evaluates whether the level of blood glucose is an appropriate value, that is, whether the level of blood glucose is measured at an appropriate time point, on the basis of the tendency of fluctuation in the level of glucose in the interstitial fluid in the period T associated with the level of blood glucose by the association unit 12. Hereinafter, the evaluation based on tendency of fluctuation in the level of glucose in the interstitial fluid in the period T will be referred to as a “first evaluation”. Hereinafter, a specific method of the first evaluation performed by the evaluation unit 14 will be described.

First, the evaluation unit 14 derives a tendency of fluctuation in the level of glucose in the interstitial fluid in the period T on the basis of a plurality of levels of glucose in the interstitial fluid associated with the level of blood glucose. The “tendency of fluctuation in the level of glucose in the interstitial fluid” is represented by, for example, a range of fluctuation Dx in the level of glucose in the interstitial fluid in the period T (that is, a difference between the maximum value and the minimum value of the plurality of levels of glucose in the interstitial fluid in the period T). In the examples of FIGS. 2 and 3, the evaluation unit 14 derives the range of fluctuation Dx in the level of glucose in the interstitial fluid in the period T1 as “83 to 88”, and derives the range of fluctuation Dx in the level of glucose in the interstitial fluid in the period T2 as “84 to 112”.

Next, the evaluation unit 14 estimates a tendency of fluctuation in the level of blood glucose in the period T, from the derived tendency of fluctuation in the level of glucose in the interstitial fluid, on the basis of the correlation data in which a correlation between the level of blood glucose and the level of glucose in the interstitial fluid is determined in advance. The correlation data is data generated in advance by performing an analysis on the basis of an actual result of a combination (hereinafter, simply referred to as “combination”) of the level of blood glucose and the level of glucose in the interstitial fluid at the same time points, and is, for example, stored in advance in the storage unit 22.

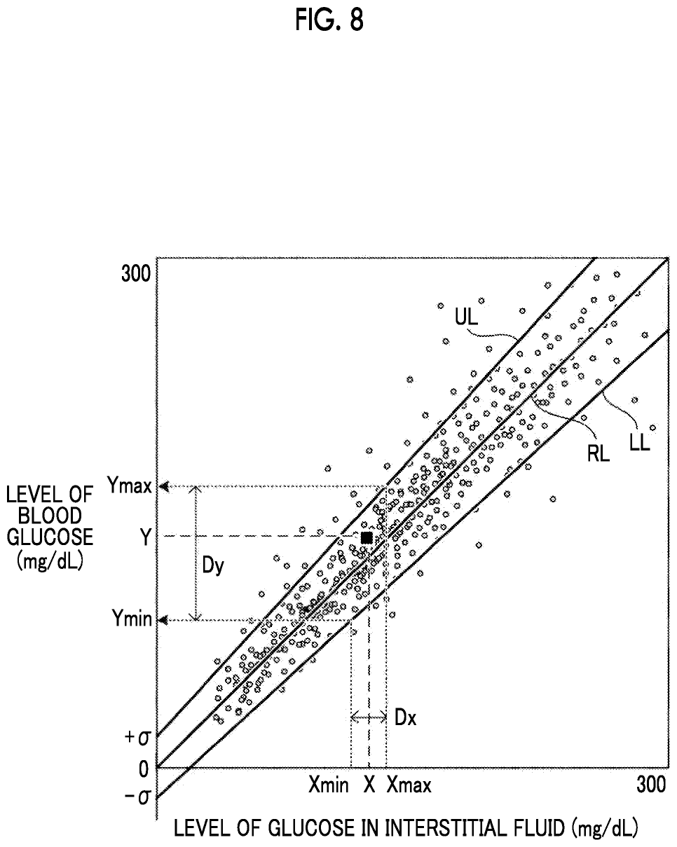

Here, the correlation data between the level of blood glucose and the level of glucose in the interstitial fluid will be described with reference to FIGS. 7 and 8. FIG. 7 is a scatter diagram in which the combination results at the same time points are plotted, where the horizontal axis represents the level of glucose in the interstitial fluid and the vertical axis represents the level of blood glucose. FIG. 7 also shows an approximate straight line RL, an estimated upper limit UL, and an estimated lower limit LL, which are generated on the basis of each combination result. The approximate straight line RL, the estimated upper limit UL, and the estimated lower limit LL are correlation data between the level of blood glucose and the level of glucose in the interstitial fluid.

As shown in FIG. 7, the combination (X, Y) of the level of glucose in the interstitial fluid (X) and the level of blood glucose (Y) is not necessarily present on the approximate straight line RL. That is, the level of blood glucose (Y) varies with respect to the level of glucose in the interstitial fluid (X). The estimated upper limit UL and the estimated lower limit LL are defined such that levels of blood glucose having variations are included in a range between the estimated upper limit UL and the estimated lower limit LL (hereinafter, referred to as “estimated interval”) with a predetermined probability.

For example, in a case where a standard deviation of the level of blood glucose with respect to the approximate straight line RL is σ, the approximate straight lines RL±σ are defined as the estimated upper limit UL and the estimated lower limit LL, respectively. Assuming that the probability distribution of the combination (X, Y) follows a normal distribution, the estimated interval includes the combination (X, Y) with probabilities of 34% above and below (68% in total) from the approximate straight line RL as a center. Consequently, assuming that the probability distribution of the newly obtained combination (X, Y) of the level of glucose in the interstitial fluid and the level of blood glucose also follows the normal distribution, it is estimated that the estimated interval includes 68% of the newly obtained combination (X, Y).

In addition, as shown in each plot in FIG. 7, the following are known. The stronger correlation between the level of blood glucose and the level of glucose in the interstitial fluid (that is, the smaller the variation) is, the smaller the level of blood glucose and the level of glucose in the interstitial fluid are. The weaker the correlation (that is, the greater the variation) is, the larger the level of blood glucose and the level of glucose in the interstitial fluid are. Therefore, as shown in FIG. 8, it is more preferable to change the slopes of the estimated upper limit UL and the estimated lower limit LL such that the estimated interval is wider as the level of blood glucose and the level of glucose in the interstitial fluid are larger.

The evaluation unit 14 estimates the tendency of fluctuation in the level of blood glucose in the period T in consideration of two factors, which include a range of fluctuation Dx in the level of glucose in the interstitial fluid in the period T and a variation of the level of blood glucose in the level of glucose in the interstitial fluid determined by the estimated upper limit UL and the estimated lower limit LL. The “tendency of fluctuation in the level of blood glucose” is represented by, for example, an estimated range of fluctuation Dy in the level of blood glucose in period T (that is, a difference between the estimated maximum value and the estimated minimum value of the levels of blood glucose in the period T). For example, the estimated maximum value Ymax of the levels of blood glucose in the period T is estimated by adding an upward variation to the maximum value Xmax of the levels of glucose in the interstitial fluid in the period T. Similarly, the estimated minimum value Ymin of the levels of blood glucose in the period T is estimated by adding a downward variation to the minimum value Xmin of the levels of glucose in the interstitial fluid in the period T.

Specifically, the evaluation unit 14 derives, as the estimated minimum value Ymin of the levels of blood glucose, the level of blood glucose at the intersection of the minimum value 83 of the level of glucose in the interstitial fluid and the estimated lower limit LL, on the basis of range of fluctuation “83 to 88” in the level of glucose in the interstitial fluid in the period T1. Further, the level of blood glucose at the intersection of the maximum value 88 of the levels of glucose in the interstitial fluid and the estimated upper limit UL is derived as the estimated maximum value Ymax of the levels of blood glucose. Similarly, the evaluation unit 14 derives, as the estimated minimum value Ymin of the levels of blood glucose, the level of blood glucose at the intersection of the minimum value 84 of the level of glucose in the interstitial fluid and the estimated lower limit LL, on the basis of range of fluctuation “84 to 112” in the level of glucose in the interstitial fluid in the period T2. Furthermore, the level of blood glucose at the intersection of the maximum value 112 of the levels of glucose in the interstitial fluid and the estimated upper limit UL is derived as the estimated maximum value Ymax of the levels of blood glucose. Hereinafter, a description will be given under the following assumption. The evaluation unit 14 derives the estimated range of fluctuation Dy in the level of blood glucose in the period T1 as 80 to 91 and derives the estimated range of fluctuation Dy in the level of blood glucose in the period T2 as 81 to 118.

Next, the evaluation unit 14 performs the first evaluation on the level of blood glucose on the basis of the estimated tendency of fluctuation in the level of blood glucose. As described above, the level of blood glucose varies in accordance with the conditions such as before meals, after meals, at rest, during exercise, after waking up, and before going to bed, even during a single day. Consequently, in a case where the level of blood glucose is accidentally measured at a timing at which the level of blood glucose is low, a comment (details will be described later) is output using a value lower than the original level of blood glucose, and the reliability thereof is lowered. Therefore, the evaluation unit 14 performs the first evaluation on the level of blood glucose by estimating whether or not the level of blood glucose is stable in the period T. For example, the evaluation unit 14 evaluates that the level of blood glucose is stable in the period T and the level of blood glucose is appropriate in a case where the estimated range of fluctuation Dy in the level of blood glucose in the period T is equal to or less than a predetermined threshold value.

For example, it is assumed that the threshold value is “15”. As described above, since the estimated range of fluctuation Dy in the level of blood glucose in the period T1 derived by the evaluation unit 14 is “11 (80 to 91 mg/dL)” and is equal to or less than the threshold value, the evaluation unit 14 at the measurement time point t1 evaluate that the level of blood glucose is appropriate. Meanwhile, since the estimated range of fluctuation Dy in the level of blood glucose in the period T2 derived by the evaluation unit 14 is “37 (81 to 118 mg/dL)” and is equal to or greater than the threshold value, the evaluation unit 14 evaluates that the level of blood glucose at the measurement time point t2 is inappropriate.

In addition, the evaluation unit 14 may perform the first evaluation of the level of blood glucose on the basis of the tendency of fluctuation in the level of glucose in the interstitial fluid. For example, in a case where the range of fluctuation Dx in the level of glucose in the interstitial fluid in the period T is equal to or greater than a predetermined threshold value, the evaluation unit 14 may evaluate that the level of blood glucose is unstable in the period T and the level of blood glucose is inappropriate. The reason for this is that, in a case where the range of fluctuation Dx in the level of glucose in the interstitial fluid in the period T is excessively large, it is possible to estimate that the level of blood glucose is inappropriate without estimating the tendency of fluctuation in the level of blood glucose.

The control unit 16 performs control for outputting the level of glucose in the interstitial fluid associated with the level of blood glucose by the association unit 12. Further, the control unit 16 performs control for outputting at least one of a comment corresponding to the first evaluation performed by the evaluation unit 14 and a comment corresponding to tendency of fluctuation in the level of blood glucose estimated by the evaluation unit 14. The “comment” is a message transmitted to the user regarding the content relating to the health examination, disease prevention, health promotion, and the like, and includes, for example, notification of the measurement result, advice and warning on the basis of the measurement result, and the like. Examples of the form of the “output” include display on the display 24, reading aloud through voice, printing using a printer, and transmission of data to an external device owned by a hospital, a testing institution, and the like.

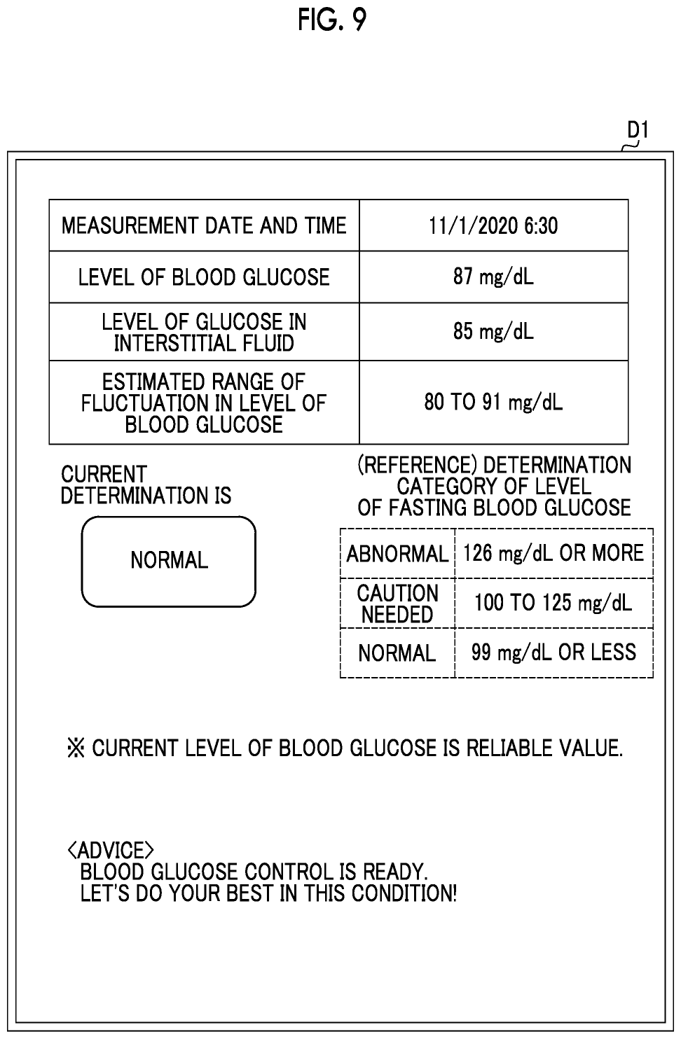

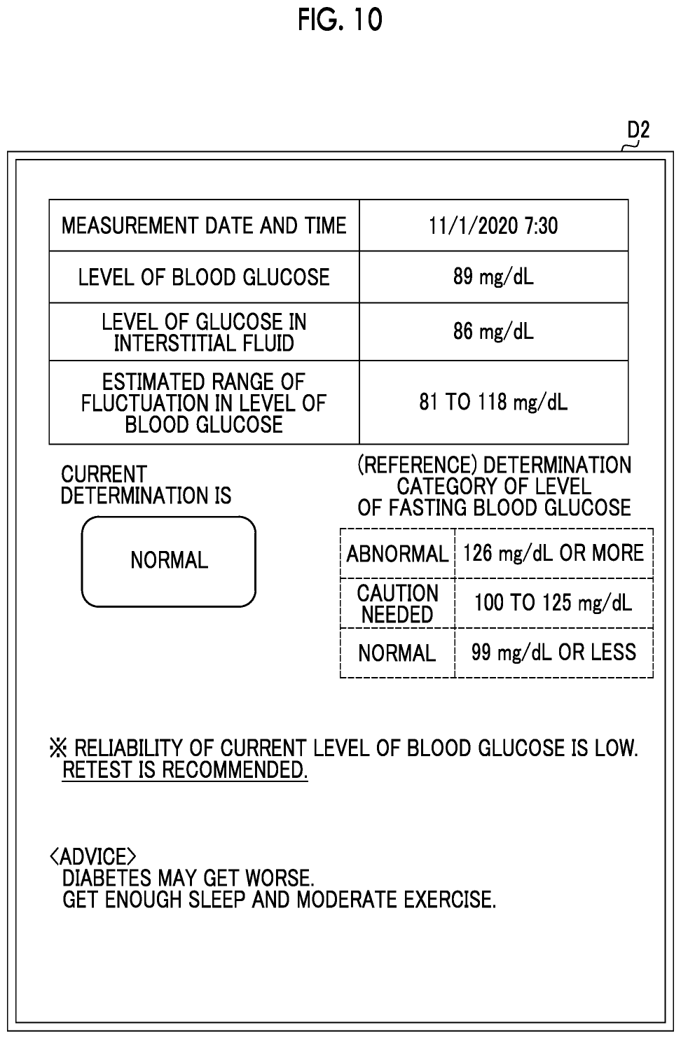

FIGS. 9 and 10 show an example of a screen displayed on the display 24 as an example of the form of output performed by the control unit 16. A screen D1 shown in FIG. 9 relates to the level of glucose in the interstitial fluid and the level of blood glucose at the measurement time point t1 (period T1). A screen D2 shown in FIG. 10 relates to the level of glucose in the interstitial fluid and the level of blood glucose at the measurement time point t2 (period T2). As shown in FIGS. 9 and 10, the control unit 16 performs control for displaying the time information (measurement dates and times), the level of blood glucose, and the level of glucose in the interstitial fluid which are acquired by the acquisition unit 10, and the estimated range of fluctuation Dy in the level of blood glucose estimated by the evaluation unit 14.

As described above, the evaluation unit 14 evaluates that the level of blood glucose at the measurement time point t1 is an appropriate value in the first evaluation. In such a case, as shown in FIG. 9, the control unit 16 performs control for displaying a comment having a content, which indicates that the level of blood glucose is appropriate, such as a message that “* The current level of blood glucose is a reliable value”. In contrast, the evaluation unit 14 evaluates that the level of blood glucose at the measurement time point t2 is an inappropriate value in the first evaluation. In such a case, as shown in FIG. 10, the control unit 16 perform control for displaying a comment having a content, in which the level of blood glucose is inappropriate and which is for recommending retest, such as a message that “* The reliability of the current level of blood glucose is low. Retest is recommended”.

Further, it is generally known that a threshold value at which a determination of diabetes using a level of fasting blood glucose is a normal determination is 99 mg/dL or less. Since the level of blood glucose at the measurement time point t1 is 87 mg/dL and the level of blood glucose at the measurement time point t2 is 89 mg/dL, both of the determinations based on the level of blood glucose are normal determinations. In such a case, as shown in FIGS. 9 and 10, the control unit 16 displays a comment indicating a result of determination based on the level of blood glucose obtained by the measurement device 3, such as a message that “the current determination is normal”.

Meanwhile, the control unit 16 outputs a comment in consideration of the estimated range of fluctuation Dy in the level of blood glucose estimated by the evaluation unit 14. For example, the maximum value of the estimated range of fluctuation Dy in the level of blood glucose in the period T1 is 91, and it can be said that normal determination is made even in consideration of the range of fluctuation. Therefore, as shown in FIG. 9, the control unit 16 displays a comment having a content such as a message that “The blood glucose is under control . . . ”. In contrast, the maximum value of the estimated range of fluctuation Dy in the level of blood glucose in the period T2 is 118, and it cannot always be said that the determination is normal in consideration of the range of fluctuation. Therefore, as shown in FIG. 10, the control unit 16 displays a comment having a content such as a message that “Diabetes may get worse . . . ”

Further, for example, the control unit 16 may store the first evaluation in the previous test in the storage unit 22 and output a comment corresponding to the first evaluation in the previous test. For example, in a case where the first evaluation is continuously evaluated as inappropriate in the previous test and the current test, a comment may be output, which includes advice and a warning for measuring the level of blood glucose at an appropriate time point for the user. Furthermore, in such a case, the determination criteria for diabetes using the level of fasting blood glucose may be changed severely.

Further, for example, the control unit 16 may output a comment of the content further recommending a test in a case where the level of blood glucose is evaluated to be inappropriate in the first evaluation. For example, a comment may be output, which has a content recommending a postprandial blood glucose test, a glucose tolerance test, an intestinal bacterium test, and the like. Furthermore, a test device necessary for the tests may be automatically reserved, or a test may be automatically reserved at a hospital, a testing institution, or the like.

Further, for example, in a case where the level of blood glucose is evaluated to be inappropriate in the first evaluation, the control unit 16 may output a comment including advice on exercise, sleep, eating, and the like for improving the level of blood glucose. The reason for this is that, in a case where the first evaluation is inappropriate, it is considered that there is room for improvement even in a case where the determination of diabetes on the basis of the level of blood glucose and the maximum value of the estimated range of fluctuation Dy in the level of blood glucose is normal. Examples of such advice include exercising after each meal, combining aerobic exercise with resistance exercise, and taking sufficient sleep time. Furthermore, the examples include determination of a meal time, taking a low glycemic index (GI) value ingredient, presentation of a recipe for a diabetic patient, order and speed of eating, and the like. Moreover, meals for diabetic patients may be automatically delivered.

Further, for example, in a case where the level of blood glucose is evaluated to be inappropriate in the first evaluation, the control unit 16 may analyze the content of the meal that a user has eaten before the measurement of the level of blood glucose and may output a comment relating to the analysis result. The contents of the meal are, for example, nutrients (for example, GI value, calories, sugar, and the like), order of eating, eating speed, and the like of what is eaten. The content of the meal may be input by, for example, the user through the input unit 25, or may be acquired by analyzing a video which is obtained by imaging a state of the meal of the user with a camera. Furthermore, a comment such as a message that “Please upload the image of the meal” may be output to urge the user to upload the image of the meal. Examples of comments relating to the results of analyzing the contents of a meal include advice such as a message that “Please eat vegetables first” and “Please eat slowly”.

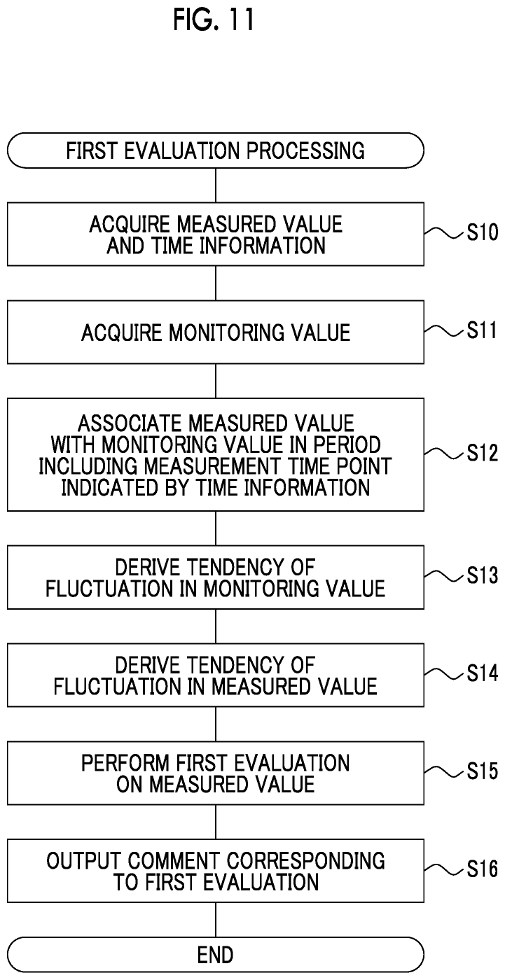

Next, an operation of the information processing apparatus 20 according to the present exemplary embodiment will be described with reference to FIG. 11. The CPU 21 executes the information processing program 27, thereby executing the first evaluation processing shown in FIG. 11. The first evaluation processing shown in FIG. 11 is executed, for example, in a case where the user issues an instruction to start the processing through the input unit 25.

In step S10 of FIG. 11, the acquisition unit 10 acquires the time information which indicates the measured value (for example, the level of blood glucose) and the measurement time point t of the measured values. In step S11, the acquisition unit 10 acquires the monitoring values (for example, the levels of glucose in the interstitial fluid) at a plurality of time points. In step S12, the association unit 12 associates the measured value acquired in step S10 with the plurality of monitoring values in the predetermined period T, which includes at least one of a time point before or a time point after the measurement time point t indicated by the time information acquired in step S10, among the monitoring values at the plurality of time points acquired in step S11.

In step S13, the evaluation unit 14 derives a tendency of fluctuation in the monitoring value in the period T on the basis of the plurality of monitoring values associated with the measured value in step S12. In step S14, the evaluation unit 14 estimates the tendency of fluctuation in the monitoring value of the measured value in the period T, from the tendency of fluctuation in the monitoring value in the period T derived in step S13, on the basis of the correlation data in which the correlation between the measured value and the monitoring values is determined in advance. In step S15, the evaluation unit 14 performs the first evaluation on the measured value, on the basis of the tendency of fluctuation in the measured value estimated in step S14. In step S16, the control unit 16 outputs a comment corresponding to the content of the first evaluation performed in step S15, and ends the first evaluation processing.

As described above, the information processing apparatus 20 according to the first exemplary embodiment includes at least one processor. The processor is configured to acquire a measured value, which is measured in a sample test, together with time information indicating a measurement time point of the measured value, acquire a monitoring value which is obtained by monitoring biological information having a correlation with the measured value, and associate the measured value with the monitoring value at the measurement time point indicated by the time information. Consequently, it is possible to evaluate whether or not the measured value is an appropriate value, on the basis of the monitoring value associated with the measured value, and it is possible to acquire an appropriate measurement result.

It should be noted that, in the first exemplary embodiment, the embodiment in which the range of fluctuation is used as a specific example of the tendency of fluctuation has been described, but the present invention is not limited thereto. For example, as the tendency of fluctuation, the slope of the approximate straight line of the level of glucose in the interstitial fluid in a plurality of periods T, the dispersion, the standard deviation, fluctuation coefficient (standard deviation/arithmetic mean value), and the like may be used.

Further, it is known that the correlation between the level of blood glucose and the level of glucose in the interstitial fluid changes in accordance with various factors. Therefore, in the first exemplary embodiment, the evaluation unit 14 may estimate the tendency of fluctuation in the level of blood glucose in the period T from the tendency of fluctuation in the level of glucose in the interstitial fluid, on the basis of a plurality of pieces of the correlation data different for each of the various factors.

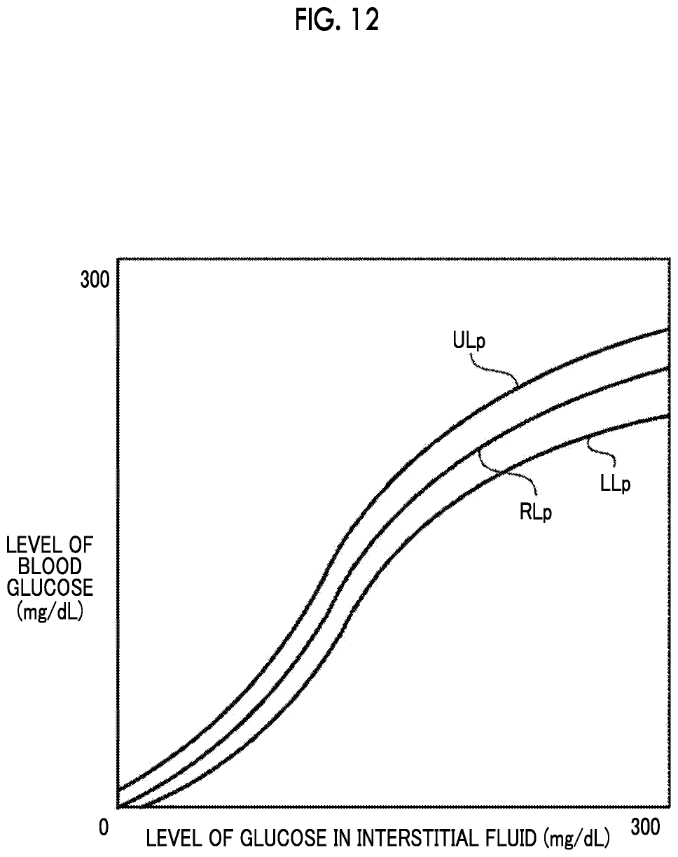

For example, as shown in FIG. 4, it is known that a level of blood glucose of a diabetic user rapidly rises and falls in a postprandial period Po (so-called blood glucose level spike). Further, it is known that the level of glucose in the interstitial fluid follows the change in the level of blood glucose with a delay of about 10 minutes. Thus, the delay is maximally the time which is obtained by adding the monitoring interval of the level of glucose in the interstitial fluid (for example, 25 minutes by adding 15 minutes to 10 minutes). Consequently, in the period during which the blood glucose level spike is lowered, as shown in FIG. 12, there is a correlation such that the level of glucose in the interstitial fluid increases with respect to the level of blood glucose. The degree of decrease of the postprandial blood glucose level spike may be used for determining diabetes. In such a case, it is preferable to use the correlation data as shown in FIG. 12. In FIG. 12, RLp is an approximation curve, where ULp is an estimated upper limit and LLp is an estimated lower limit.

Specifically, the storage unit 22 stores in advance the correlation data of the fasting period Pr (refer to FIG. 4) and the correlation data of the postprandial period Po (refer to FIG. 12). The acquisition unit 10 acquires timing information which indicates whether the measurement time point t at which the level of blood glucose is measured is fasting or postprandial. The evaluation unit 14 estimates the tendency of fluctuation in the level of blood glucose in the period T, from the tendency of fluctuation in the level of glucose in the interstitial fluid, on the basis of the correlation data corresponding to the timing indicated by the timing information among the correlation data corresponding to fasting and postprandial, respectively. The timing information may be input by, for example, the user through the input unit 25, or the meal time may be set in advance, and the timing information may be determined in accordance with the time information.

Further, for example, it is known that the degree of increase and the degree of decrease in the level of blood glucose change in accordance with the contents of the meal such as the nutrients (for example, GI value, calories, sugars, and the like), the order of eating, and the eating speed of the food to be eaten. For example, in a case of glucose sugar having a high GI value, the level of blood glucose changes rapidly, and in a case of fructose having a low GI value, the level of blood glucose changes more slowly than glucose sugar. As described above, the level of glucose in the interstitial fluid follows the change in the level of blood glucose with a delay. Therefore, variation in the level of blood glucose with respect to the level of the glucose in the interstitial fluid also changes between the meal in which the level of blood glucose changes rapidly and the meal in which the level of blood glucose changes slowly.

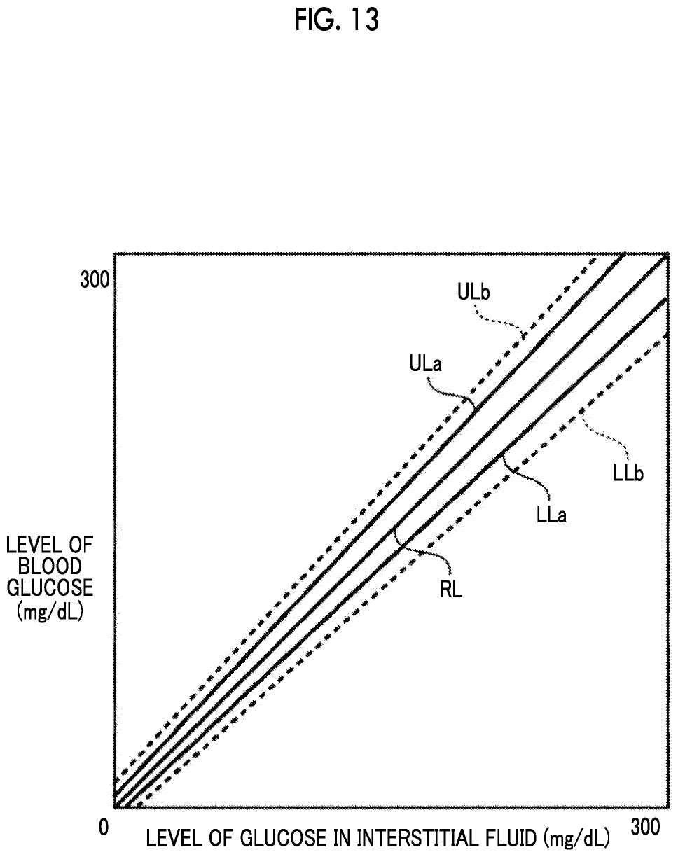

Therefore, as shown in FIG. 13, it is preferable that the storage unit 22 stores in advance the correlation data that differs in accordance with the content of the meal. The acquisition unit 10 acquires meal information indicating a content of a meal that a person who measures the level of blood glucose has eaten before the measurement of the level of blood glucose. The evaluation unit 14 estimates the tendency of fluctuation in the level of blood glucose in the period T, from the tendency of fluctuation in the level of glucose in the interstitial fluid, on the basis of the correlation data corresponding to the content of the meal indicated by the meal information among the plurality of pieces of the correlation data different for each content of the meal. For example, in a case of the content of the meal in which the level of blood glucose changes slowly, the estimated upper limit ULa and the estimated lower limit LLa, in which the estimated interval in FIG. 13 is narrow (that is, the variation is small), are used. In contrast, in a case of the content of the meal in which the level of blood glucose changes rapidly, the estimated upper limit ULb and the estimated lower limit LLb, in which the estimated interval in FIG. 13 is wide (that is, the variation is large), are used. The meal information may be input by, for example, through the input unit 25, or may be acquired by analyzing a video which is obtained by capturing a state of the user's meal with a camera.

Further, in the correlation data, there may be a range in which the variation in the level of glucose in the interstitial fluid of the level of blood glucose is equal to or greater than a predetermined threshold value (that is, the variation is large). In such a case, the evaluation unit 14 may perform the first evaluation that the level of blood glucose located in the range is inappropriate. The reason for this is that, in a range in which the correlation between the level of blood glucose and the level of glucose in the interstitial fluid is weak, the accuracy is lowered even in a case where the tendency of fluctuation in the level of blood glucose is estimated on the basis of the level of glucose in the interstitial fluid.

Further, in a first exemplary embodiment, the embodiment in which the level of glucose in the interstitial fluid is used as the biological information having a correlation with the level of blood glucose has been described, but the present invention is not limited thereto. As biological information having a correlation with the level of blood glucose, a level of glucose included in sweat or saliva, an electrocardiographic signal, a blood pressure, a body temperature, and the like are also known. In a case where the values are used instead of the levels of glucose in the interstitial fluid, the evaluation unit 14 estimates the tendency of fluctuation in the level of blood glucose, on the basis of the monitoring values of the above-mentioned values and the correlation data in which the correlation between the monitoring values and the level of blood glucose is predetermined. Furthermore, the evaluation unit 14 may perform the first evaluation by appropriately combining a plurality of types of monitoring values of a part or all of the values including the level of glucose in the interstitial fluid. Moreover, the evaluation unit 14 may use the correlation data which is obtained by performing big data analysis (for example, deep learning by artificial intelligence (AI)) based on optional biological information obtained by the monitoring device 4 and the level of blood glucose in addition to the above-mentioned values.

### SECOND EXEMPLARY EMBODIMENT

In the first exemplary embodiment, the first evaluation is performed on the basis of the tendency of fluctuation in the level of glucose in the interstitial fluid in the period T in consideration of the fluctuation in the level of blood glucose during a single day. Meanwhile, it is known that the level of blood glucose may fluctuate as compared with other days depending on a physical condition, an amount of activity, a sleeping time, a content of the meal, and the like on that day. By also taking into account this daily fluctuation, it is possible to more accurately evaluate whether the level of blood glucose is an appropriate value (whether the result is better or worse than usual), that is, whether the level of blood glucose is measured at an appropriate time point.

Therefore, the information processing apparatus 20 according to the present exemplary embodiment compares the level of glucose in the interstitial fluid at the measurement time point t at which the level of blood glucose is measured with the reference value in which the daily fluctuation is taken into consideration, thereby evaluating whether the level of blood glucose is an appropriate value, that is, whether the level of blood glucose is measured at an appropriate time point. Hereinafter, the evaluation in consideration of the daily fluctuation will be referred to as a “second evaluation”. Hereinafter, an example of the configuration of the information processing apparatus 20 according to the present exemplary embodiment will be described. However, in the present exemplary embodiment, the description, which overlaps with the first exemplary embodiment, will be omitted.

As described above, the acquisition unit 10 acquires the level of blood glucose and the time information which indicates the measurement time point t of the level of blood glucose, from the measurement device 3. Further, the acquisition unit 10 acquires the level of glucose in the interstitial fluid at a plurality of time points from the monitoring device 4. The association unit 12 associates the level of blood glucose acquired by the acquisition unit 10 with the level of glucose in the interstitial fluid at the measurement time point t indicated by the time information.

First, the evaluation unit 14 derives a deviation between a reference value of the level of glucose in the interstitial fluid at the measurement time point t indicated by the time information and the level of glucose in the interstitial fluid at the measurement time point t indicated by the time information.

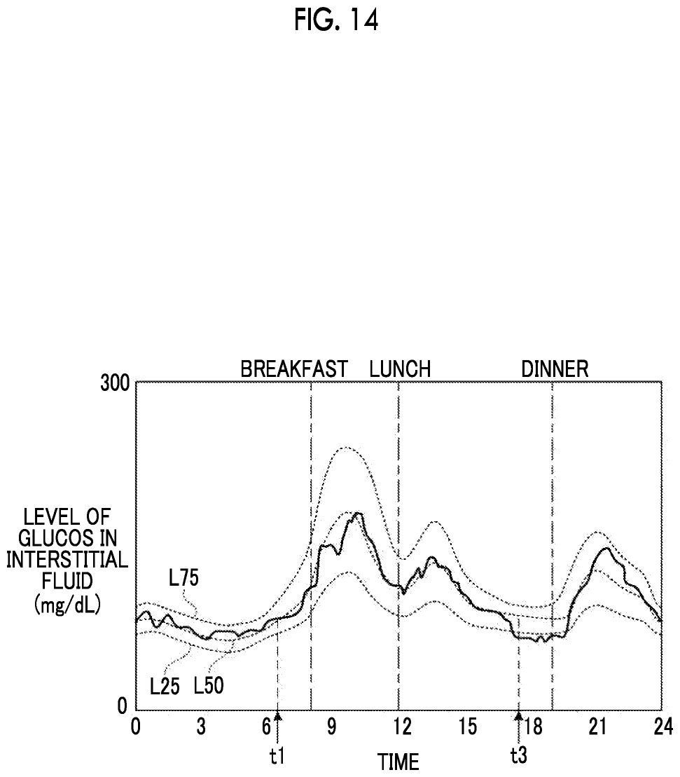

Here, the reference value will be described with reference to FIG. 14. FIG. 14 is a graph in which the horizontal axis represents time and the vertical axis represents the level of glucose in the interstitial fluid. In FIG. 14, the level of glucose in the interstitial fluid on a certain day is shown by the solid line, and the reference lines L50, L25, and L75 are shown by the broken line. The reference lines L50, L25, and L75 are respectively derived by analyzing the daily levels of glucose in the interstitial fluid monitored for the same person. The reference line L50 indicates a representative value of the level of glucose in the interstitial fluid for each day at each time point. The “representative value” is, for example, an arithmetic mean value, a median value, a mode value, or the like. The reference line L25 and the reference line L75 are defined such that the level of glucose in the interstitial fluid is included between the reference line L25 and the reference line L75 with probabilities of 25% above and below the reference line L50 (50% in total).

The evaluation unit 14 acquires a reference value of the level of glucose in the interstitial fluid at the measurement time point t indicated by the time information on the basis of the reference line L50, and derives a deviation from the level of glucose in the interstitial fluid. For example, the evaluation unit 14 derives a deviation at the measurement time point t1 shown in FIG. 14 as 0. Further, a deviation at the measurement time point t3 shown in FIG. 14 is derived as 20.

Next, the evaluation unit 14 performs the second evaluation on the level of blood glucose on the basis of the derived deviation. As described above, the level of blood glucose fluctuates day by day. Consequently, in a case where the measurement is performed on a day in a case where the level of blood glucose is accidentally low, the comment is output using a value lower than the level of blood glucose on most of the days, and the validity thereof is lowered. Therefore, the evaluation unit 14 performs the second evaluation on the level of blood glucose in accordance with whether or not the deviation from the reference value of the level of glucose in the interstitial fluid at the measurement time point t of the level of blood glucose is allowable. For example, in a case where the deviation of the level of glucose in the interstitial fluid at the measurement time point t is equal to or less than a predetermined threshold value, the evaluation unit 14 sets the level of blood glucose at the measurement time point t to the same value as that of the other day, and evaluates that the level of blood glucose is appropriate.

For example, it is assumed that the threshold value of the deviation is “15”. As described above, the deviation at the measurement time point t1 derived by the evaluation unit 14 is 0 and is equal to or less than the threshold value. Therefore, the evaluation unit 14 evaluates that the level of blood glucose at the measurement time point t1 is appropriate. Meanwhile, the deviation at the measurement time point t3 derived by the evaluation unit 14 is 20 and is equal to or greater than the threshold value. Therefore, the evaluation unit 14 evaluates that the level of blood glucose at the measurement time point t3 is inappropriate.

The control unit 16 performs control for outputting a comment corresponding to the second evaluation performed by the evaluation unit 14. For example, in a case where the level of blood glucose is evaluated to be inappropriate in the second evaluation, the control unit 16 outputs a comment, which recommends the test on another day, such as a message that “Are you feeling tired today? Let's test again tomorrow”. Further, the control unit 16 may output the same comments as in the first evaluation described in the first exemplary embodiment in accordance with the second evaluation.

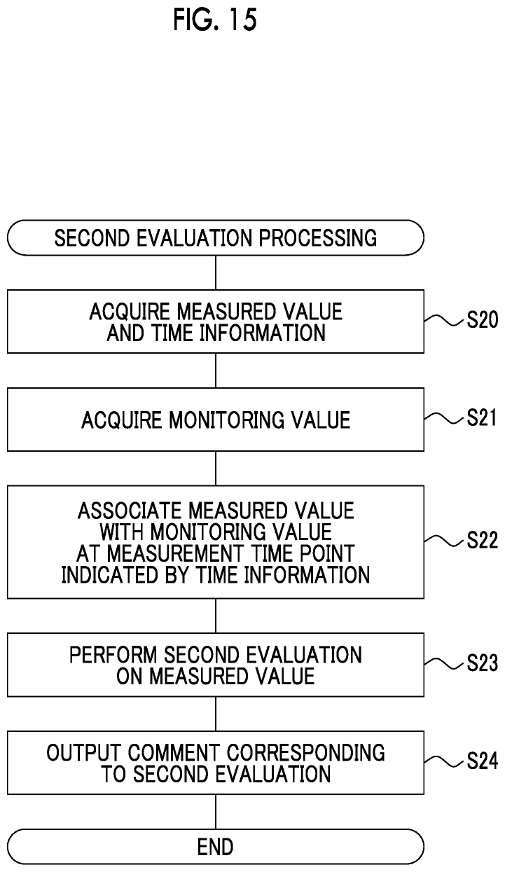

Next, an operation of the information processing apparatus 20 according to the present exemplary embodiment will be described with reference to FIG. 15. The CPU 21 executes the information processing program 27, thereby executing the second evaluation processing shown in FIG. 15. The second evaluation processing shown in FIG. 15 is executed, for example, in a case where a user issues an instruction to start the processing through the input unit 25.

In step S20 of FIG. 15, the acquisition unit 10 acquires the time information which indicates the measured value (for example, the level of blood glucose) and the measurement time point t of the measured values. In step S21, the acquisition unit 10 acquires the monitoring value (for example, the level of glucose in the interstitial fluid). In step S22, the association unit 12 associates the measured value, which is acquired in step S20, with the monitoring value at the measurement time point t indicated by the time information acquired in step S20 among the monitoring values acquired in step S21.

In step S23, the evaluation unit 14 performs the second evaluation on the measured value, on the basis of the deviation between the reference value of the monitoring value at the measurement time point t indicated by the time information acquired in step S20 and the monitoring value associated with the measured value in step S22. In step S24, the control unit 16 outputs a comment corresponding to the content of the second evaluation performed in step S23, and ends the second evaluation processing.

As described above, the information processing apparatus 20 according to the second exemplary embodiment performs the second evaluation on the measured value, on the basis of the deviation between the reference value of the monitoring value at the measurement time point t indicated by the time information and the monitoring value at the measurement time point t indicated by the time information. Consequently, it is possible to evaluate whether or not the measured value is an appropriate value on the basis of the monitoring value (that is, the monitoring value associated with the measured value) at the measurement time point t. As a result, it is possible to acquire an appropriate measurement result.

It should be noted that, in the second exemplary embodiment, unlike the first exemplary embodiment, the acquisition unit 10 does not necessarily have to acquire the level of glucose in the interstitial fluid at a plurality of time points. For example, the acquisition unit 10 may acquire only one level of glucose in the interstitial fluid corresponding to the measurement time point t of the level of blood glucose, and the evaluation unit 14 may perform the second evaluation on the level of blood glucose on the basis of the level of glucose in the interstitial fluid. However, in order to more appropriately perform the second evaluation, it is more preferable to perform the second evaluation on the level of blood glucose, on the basis of a plurality of levels of glucose in the interstitial fluid in a period T including at least one of time points before or after the measurement time point t of the level of blood glucose.

Further, in the second exemplary embodiment, the evaluation unit 14 may perform the second evaluation in accordance with whether or not the level of glucose in the interstitial fluid at the measurement time point t indicated by the time information is included between the reference line L25 and the reference line L75.

Further, in the second exemplary embodiment, the evaluation unit 14 may change the threshold value of the deviation depending on the time, the timing of fasting or postprandial, and the like. The reason for this is that, as shown in FIG. 14, it is known that the level of postprandial blood glucose varies from day to day more than the level of fasting blood glucose.

### THIRD EXEMPLARY EMBODIMENT

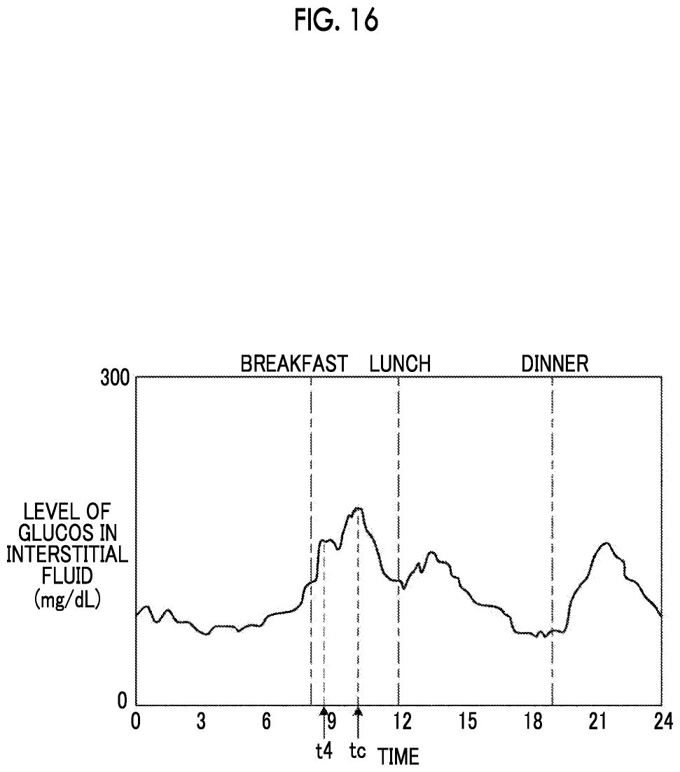

In some cases, a peak level of blood glucose or the like in the postprandial blood glucose level spike is used for determining diabetes. For example, in the example of FIG. 16, in a case where it is desired to measure a peak level of blood glucose in a blood glucose level spike after breakfast, it is preferable that the level of blood glucose is measured at a target time point tc as a peak. However, in practice, it is difficult to grasp in advance the target time point tc at which the level of blood glucose reaches the peak value, and the measurement time point of the level of blood glucose may deviate from the target time point tc.

Therefore, the information processing apparatus 20 according to the present exemplary embodiment evaluates whether it is possible to allow the deviation between the measurement time point t and the target time point tc on the basis of the value of the level of glucose in the interstitial fluid at the measurement time point t of the level of blood glucose, that is, whether the measurement time point t is an appropriate time point. Hereinafter, the evaluation will be referred to as a “third evaluation”. Hereinafter, an example of the configuration of the information processing apparatus 20 according to the present exemplary embodiment will be described. However, in the present exemplary embodiment, the description, which overlaps with the first and second exemplary embodiments, will be omitted.

For example, a description will be given under the assumption that the actual measurement of the level of blood glucose is performed at a measurement time point t4 with respect to the target time point tc in FIG. 16. As described above, the acquisition unit 10 acquires the level of blood glucose and the time information indicating the measurement time point t4 of the level of blood glucose from the measurement device 3. Further, the acquisition unit 10 acquires the level of glucose in the interstitial fluid, from the monitoring device 4 at a plurality of time points including at least a time point corresponding to the measurement time point t4 and the target time point tc. The association unit 12 associates the level of blood glucose, which is acquired by the acquisition unit 10, with the level of glucose in the interstitial fluid at the measurement time point t4 indicated by the time information.

The evaluation unit 14 performs the third evaluation at the measurement time point t4 indicated by the time information, on the basis of the level of glucose in the interstitial fluid associated with the level of blood glucose. Specifically, first, the evaluation unit 14 specifies the level of glucose in the interstitial fluid (in such a case, the peak value) at the target time point tc, on the basis of the levels of glucose in the interstitial fluid at the plurality of time points acquired by the acquisition unit 10. Next, the evaluation unit 14 derives a difference between the level of glucose in the interstitial fluid at the measurement time point t4 and the level of glucose in the interstitial fluid at the target time point tc which is associated with the level of blood glucose. Next, in a case where the derived difference is equal to or less than a predetermined threshold value, the evaluation unit 14 is able to allow the difference between the level of glucose in the interstitial fluid at the measurement time point t4 and the level of glucose in the interstitial fluid at the target time point tc, and evaluates that the measurement time point t is an appropriate time point.

The control unit 16 performs control for outputting a comment corresponding to the third evaluation performed by the evaluation unit 14. For example, in a case where it is evaluated that the measurement time point t4 is inappropriate in the third evaluation, the control unit 16 outputs a comment including a warning such as a message that “The level of blood glucose could not be measured at the target time point”. Further, the control unit 16 may output the same comments as in the first evaluation described in the first exemplary embodiment in accordance with the third evaluation.

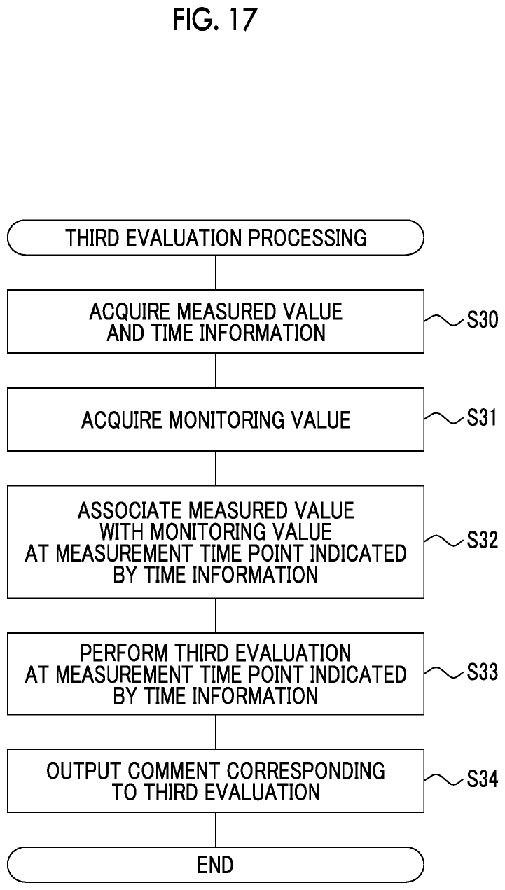

Next, an operation of the information processing apparatus 20 according to the present exemplary embodiment will be described with reference to FIG. 17. The CPU 21 executes the information processing program 27, thereby executing the third evaluation processing shown in FIG. 17. The third evaluation processing shown in FIG. 17 is executed, for example, in a case where a user issues an instruction to start the processing through the input unit 25.

In step S30 of FIG. 17, the acquisition unit 10 acquires the time information which indicates the measured value (for example, the level of blood glucose) and the measurement time point t of the measured value. In step S31, the acquisition unit 10 acquires the monitoring value (for example, the level of glucose in the interstitial fluid). In step S32, the association unit 12 associates the measured value, which is acquired in step S30, with the monitoring value at the measurement time point t indicated by the time information acquired in step S30 among the monitoring values acquired in step S31.

In step S33, the evaluation unit 14 performs the third evaluation on the measured value, on the basis of the monitoring value associated with the measured value in step S32. In step S34, the control unit 16 outputs a comment corresponding to the content of the third evaluation performed in step S33, and ends the third evaluation processing.

As described above, the information processing apparatus 20 according to the third exemplary embodiment performs the third evaluation for the measurement time point t indicated by the time information, on the basis of the monitoring value associated with the measured value. Consequently, it is possible to evaluate whether the measurement time point t of the measured value is an appropriate time point on the basis of the monitoring value associated with the measured value, and it is possible to acquire an appropriate measurement result.

It should be noted that, in the third exemplary embodiment, unlike the first exemplary embodiment, the acquisition unit 10 does not necessarily have to acquire the level of glucose in the interstitial fluid at a plurality of time points. For example, the level of glucose in the interstitial fluid at the target time point tc may be estimated in advance, on the basis of the actual results of the level of blood glucose or the level of glucose in the interstitial fluid before the date of the test. In such a case, the acquisition unit 10 acquires only one level of glucose in the interstitial fluid corresponding to the measurement time point t4 of the level of blood glucose, and the evaluation unit 14 performs the third evaluation, on the basis of the current level of glucose in the interstitial fluid and the previously estimated level of glucose in the interstitial fluid.

Further, in the third exemplary embodiment, the evaluation unit 14 may perform the third evaluation, on the basis of the temporal divergence between the target time point tc and the measurement time point t4 indicated by the time information. In such a case, for example, the evaluation unit 14 derives a temporal difference between the target time point tc and the measurement time point t4, and evaluates that the measurement time point t4 is an appropriate time point in a case where the temporal difference is equal to or less than a predetermined threshold value.

Further, in the third exemplary embodiment, the example, in which the target time point tc is the time point at which the level of glucose in the interstitial fluid reaches the peak value, has been described, but the present invention is not limited thereto. A level of blood glucose at each time point after a meal (for example, after 30 minutes, after 1 hour, after 2 hours, or the like) may be used for determining diabetes. Furthermore, for the purpose of grasping fluctuation in the level of blood glucose from day to day, the level of blood glucose may be measured at the same time every day. In such a case, as the target time point tc, for example, a time point at which a predetermined time has elapsed after the meal (for example, 30 minutes, 1 hour, 2 hours, and the like after the meal), the predetermined time, or the like may be set.

### FOURTH EXEMPLARY EMBODIMENT

In the above-mentioned first to third exemplary embodiments, the form, in which various evaluations are performed on the measured level of blood glucose and the measurement time point t of the level of blood glucose after the level of blood glucose is measured, has been described. The information processing apparatus 20 according to the present exemplary embodiment has a function of applying the disclosed technique to the first to third exemplary embodiments to recommend the measurement timing of the level of blood glucose before the measurement of the level of blood glucose. Hereinafter, an example of the configuration of the information processing apparatus 20 according to the present exemplary embodiment will be described. However, in the present exemplary embodiment, the description, which overlaps with the first to third exemplary embodiments, will be omitted.

For example, by applying the disclosed technique to the second exemplary embodiment, comparing the level of glucose in the interstitial fluid at the current time point tr in which the level of blood glucose is to be measured with the reference value in which the daily fluctuation is taken into consideration, it may be determined whether or not to recommend the measurement of the level of blood glucose. Specifically, it may be recommended to measure the level of blood glucose in a case where the deviation from the reference value of the level of glucose in the interstitial fluid at the current time point tr is allowable.

In such a case, the acquisition unit 10 acquires the level of glucose in the interstitial fluid at the current time point tr from the monitoring device 4. The evaluation unit 14 evaluates that the level of blood glucose is measurable in a case where the deviation between the reference value of the level of glucose in the interstitial fluid at the current time point tr and the level of glucose in the interstitial fluid is smaller than a predetermined threshold value.

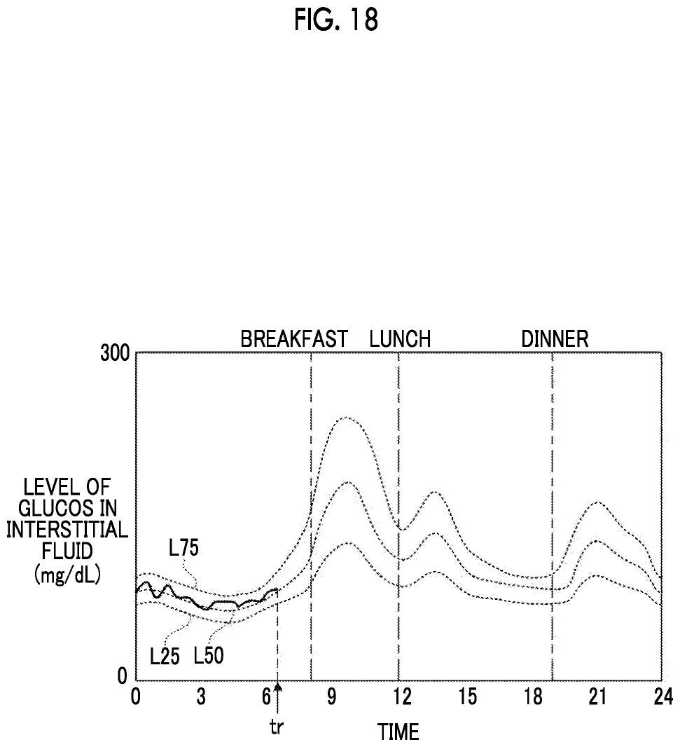

In a case where the evaluation unit 14 evaluates that the level of blood glucose is measurable, the control unit 16 performs control for recommending the measurement of the level of blood glucose. For example, the control unit 16 outputs a comment that “Currently, the level of blood glucose is moving normally. Let's measure the level of blood glucose”. Meanwhile, in a case where the evaluation unit 14 evaluates that the level of blood glucose cannot be measured, the control unit 16 performs control for issuing a warning. For example, the control unit 16 outputs a comment that “Currently, the level of blood glucose is moving abnormally. We recommend that you measure your level of blood glucose later, measure it multiple times, or stop this measurement”. Further, the control unit 16 may output a diagram as shown in FIG. 18 such that the user is able to grasp a relationship between the level of glucose in the interstitial fluid and the reference value at the current time point tr.

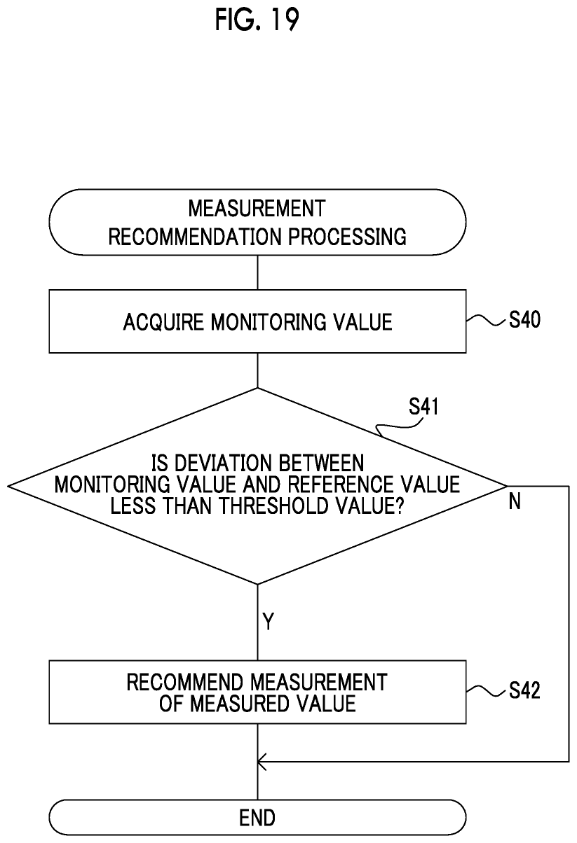

Next, an operation of the information processing apparatus 20 according to the present exemplary embodiment will be described with reference to FIG. 19. The CPU 21 executes the information processing program 27, thereby executing the measurement recommendation processing shown in FIG. 19. The measurement recommendation processing shown in FIG. 19 is, for example, an application of the disclosed technique to the second exemplary embodiment. The measurement recommendation processing shown in FIG. 19 is executed, for example, in a case where a user issues an instruction to start the processing through the input unit 25.

In step S40 of FIG. 19, the acquisition unit 10 acquires the monitoring value at the current time point (for example, the level of glucose in the interstitial fluid). In step S41, the evaluation unit 14 determines whether the deviation between the monitoring value acquired in step S40 and the reference value of the monitoring value at the current time point is less than the threshold value. In a case where a positive determination is made in step S41, the processing proceeds to step S42, and the control unit 16 performs processing of recommending measurement of the measured value. In a case where the negative determination is made in step S41 and in a case where step S42 is completed, the current measurement recommendation processing ends.

As described above, the information processing apparatus 20 according to the fourth exemplary embodiment includes at least one processor, in which the processor may be configured to acquire a monitoring value which is obtained by monitoring biological information having a correlation with a measured value which is measured in a sample test, and recommend measurement of the measured value in a case where a deviation between the monitoring value and a reference value of the monitoring value is smaller than a predetermined threshold value. Consequently, the measured value is measurable at a time point at which it is estimated that the measured value takes an appropriate value on the basis of the monitoring value. Therefore, an appropriate measurement result can be acquired.

It should be noted that, in the above-mentioned third exemplary embodiment, the measurement recommendation processing, in which the disclosed technique is applied to the second exemplary embodiment, has been described. However, the present invention is not limited thereto, and it is also possible to apply the disclosed technique to the first and third exemplary embodiments. For example, by applying the disclosed technique to the first exemplary embodiment, it may be determined whether or not to recommend the measurement of the level of blood glucose, on the basis of the tendency of fluctuation in the plurality of levels of glucose in the interstitial fluid in the predetermined period T including a time point before the current time point tr at which the level of blood glucose is to be measured. Specifically, it may be recommended to measure the level of blood glucose in a case where the tendency of fluctuation in the level of glucose in the interstitial fluid in the period T is allowable.

For example, a case where the range of fluctuation is used as the tendency of fluctuation in the level of glucose in the interstitial fluid in the period T will be described. In such a case, the acquisition unit 10 acquires a plurality of levels of glucose in the interstitial fluid from the monitoring device 4 in the predetermined period T including a time point before the current time point tr. The evaluation unit 14 derives a range of fluctuation of the plurality of levels of glucose in the interstitial fluid in a predetermined period T including a time point before the current time point tr, and evaluates that the level of blood glucose is measurable in a case where the range of fluctuation is smaller than a predetermined threshold value.

Further, for example, by applying the disclosed technique to the third exemplary embodiment, it may be determined whether to recommend measurement of the level of blood glucose in accordance with whether or not a deviation between the current time point tr and the target time point tc at which the level of blood glucose is to be measured is allowable.

For example, the level of glucose in the interstitial fluid at the target time point tc, at which the level of blood glucose is desired to be measured, may be estimated in advance, on the basis of the actual results of the level of blood glucose or the level of glucose in the interstitial fluid before the date of the test. In such a case, the acquisition unit 10 acquires the level of glucose in the interstitial fluid at the current time point tr from the monitoring device 4. The evaluation unit 14 derives a difference between the level of glucose in the interstitial fluid at the current time point tr and the level of glucose in the interstitial fluid at the pre-estimated target time point tc, and evaluates that the level of blood glucose is measurable in a case where the difference is smaller than a predetermined threshold value.

Further, for example, as the target time point tc for measuring the level of blood glucose, a time point at which a predetermined time has elapsed from the meal (for example, 30 minutes, 1 hour, 2 hours, and the like after the meal), the predetermined time, and the like may be specified. In such a case, the evaluation unit 14 derives a temporal difference between the target time point tc and the current time point tr, and evaluates that the level of blood glucose is measurable in a case where the temporal difference is equal to or less than the predetermined threshold value.

In each of the above-mentioned exemplary embodiments, an example, in which the level of glucose in the interstitial fluid is used as the monitoring value of the biological information having a correlation with the level of blood glucose, has been described, but the present invention is not limited thereto. As a monitoring value of biological information having a correlation with a level of blood glucose, it is possible to apply at least one of a level of glucose, an electrocardiographic signal, a blood pressure, and a body temperature included in sweat or saliva.

Further, in each of the above-mentioned exemplary embodiments, a form, in which an apparatus for measuring the level of glucose in the interstitial fluid is used as the monitoring device 4 using a needle-shaped filament inserted subcutaneously of the user, has been described, but the present invention is not limited thereto. For example, a wearable terminal such as a wristwatch type, a glasses type, an earphone type, or a ring type may be applied as the monitoring device 4. The wearable terminal may derive the level of glucose by analyzing a signal emitted by glucose in blood, for example, by irradiating the skin of the user with infrared rays. Furthermore, for example, at least one of the electrocardiographic signal, the blood pressure, and the body temperature of the user, which is measured by a sensor included in the wearable terminal, may be used as the monitoring value. In a case where the wearable terminal is applied, the monitoring value is measurable in a non-invasive manner. Therefore, pain at the time of puncturing, running cost, and the like can be suppressed, and the burden on the user can be reduced.

The wearable terminal is, for example, a computer such as a smart watch, and includes a CPU, a non-volatile storage unit implemented by a storage medium such as an HDD, an SSD, and a flash memory, and a memory as a temporary storage region. Further, the wearable terminal includes an input/output unit such as a button, a display, and a touch panel, and a network I/F that performs wired or wireless communication between the information processing apparatus 20 and an external network (not shown in the drawing).

Further, in each of the above-mentioned exemplary embodiments, a part or all of the measurement device 3, the monitoring device 4, and the information processing apparatus 20 may be configured as one device or a plurality of devices. For example, the measurement device 3 and the monitoring device 4 may be an integrated device. Furthermore, for example, the wearable terminal may be applied as the monitoring device 4, and the wearable terminal may have the function of the information processing apparatus 20.

Further, in each of the above-mentioned exemplary embodiments, the level of blood glucose is used as an example of the measured value, and the level of glucose in the interstitial fluid is used as an example of the monitoring value. However, the present invention is not limited thereto. For example, a blood pressure measured by a blood pressure monitor may be used as the measured value, and a blood pressure equivalent value measured by the sensor of the wearable terminal may be applied as the monitoring value. Furthermore, for example, a body temperature measured by a thermometer may be used as the measured value, and a body temperature equivalent value measured by the sensor of the wearable terminal may be applied as the monitoring value.

Further, in each of the above-mentioned exemplary embodiments, the form, in which the acquisition unit 10 acquires the measured value, the time information, and the monitoring value from the measurement device 3 and the monitoring device 4, has been described, but the present invention is not limited thereto. For example, the measurement device 3 and the monitoring device 4 may transmit the measured value, the time information, and the monitoring value to an optional aggregation server, and the acquisition unit 10 may acquire the measured value, the time information, and the monitoring value from the aggregation server. Furthermore, for example, the user may input the measured value, the time information, and the monitoring value through the input unit 25, and the acquisition unit 10 may acquire the measured value, the time information, and the monitoring value which are input by the user.

Further, in each of the above-mentioned exemplary embodiments, for example, various processors described below can be used as a hardware structure of a processing unit that executes various types of processing such as the acquisition unit 10, the association unit 12, the evaluation unit 14, and the control unit 16. As described above, various processors include not only a CPU as a general-purpose processor which functions as various processing units by executing software (programs) but also a programmable logic device (PLD) as a processor capable of changing a circuit configuration after manufacturing a field programmable gate array (FPGA); and a dedicated electrical circuit as a processor, which has a circuit configuration specifically designed to execute specific processing, such as an application specific integrated circuit (ASIC).

One processing unit may be configured as one of the various processors, or may be configured as a combination of two or more of the same or different kinds of processors (for example, a combination of a plurality of FPGAs or a combination of a CPU and an FPGA). Further, the plurality of processing units may be composed of one processor.

As an example of the plurality of processing units composed of one processor, first, as represented by computers such as a client and a server, there is a form in which one processor is composed of a combination of one or more CPUs and software and this processor functions as a plurality of processing units. Second, as represented by a system on chip (SoC), there is a form in which a processor that realizes the functions of the whole system including a plurality of processing units with a single integrated circuit (IC) chip is used. As described above, the various processing units are configured by using one or more of the various processors as a hardware structure.

Furthermore, as the hardware structure of the various processors, more specifically, it is possible to use an electric circuit (circuitry) in which circuit elements such as semiconductor elements are combined.

Further, in each of the above-mentioned exemplary embodiments, the aspect, in which the information processing program 27 is stored (installed) in the storage unit 22 in advance, has been described, but the present invention is not limited thereto. The information processing program 27 may be provided in a form in which the programs are stored in a storage medium such as a compact disc read only memory (CD-ROM), a digital versatile disc read only memory (DVD-ROM), and a universal serial bus (USB) memory. Furthermore, the information processing program 27 may be downloaded from an external device via a network. Moreover, the technique of the present disclosure extends to a storage medium that stores the information processing program non-temporarily, in addition to the information processing program.

In the technique of the present disclosure, it is also possible to appropriately combine the above-mentioned embodiments. The contents described and illustrated above are detailed descriptions of the parts relating to the technique of the present disclosure, and are merely examples of the technique of the present disclosure. For example, the above description of the configuration, function, effect, and advantage is an example of the configuration, function, effect, and advantage of a portion relating to the technique of the present disclosure. Therefore, it is needless to say that unnecessary parts may be deleted, new elements may be added, or replacements may be made in the described contents and illustrated contents shown above without departing from the scope of the technique of the present disclosure.

According to another aspect of the present disclosure, there is provided an information processing method comprising: acquiring a measured value, which is measured in a sample test, together with time information indicating a measurement time point of the measured value; acquiring a monitoring value which is obtained by monitoring biological information having a correlation with the measured value; and associating the measured value with the monitoring value at the measurement time point indicated by the time information.

The disclosure of JP2020-199170, filed on Nov. 30, 2020, is incorporated herein by reference in its entirety. All documents, patent applications, and technical standards described in the present specification are incorporated into the present specification by reference to the same extent as in a case where the individual documents, patent applications, and technical standards were specifically and individually stated to be incorporated by reference.

What is claimed is:

1. An information processing apparatus comprising at least one processor, wherein the at least one processor is configured to:
acquire a measured value, which is measured in a sample test, together with time information indicating a measurement time point of the measured value;
acquire a monitoring value which is obtained by monitoring biological information having a correlation with the measured value; and
associate the measured value with the monitoring value at the measurement time point indicated by the time information.

2. The information processing apparatus according to claim 1, wherein the at least one processor is configured to associate the acquired measured value with the monitoring value at the measurement time point indicated by the time information, after the measured value and the time information are acquired.

3. The information processing apparatus according to claim 1, wherein the at least one processor is configured to:
acquire the monitoring value at each of a plurality of time points; and
associate the measured value with a plurality of the monitoring values in a predetermined period, which includes at least one of a time point before or a time point after the measurement time point indicated by the time information, among the monitoring values at the plurality of time points.

4. The information processing apparatus according to claim 3, wherein the at least one processor is configured to:
derive a tendency of fluctuation in the monitoring value in the period on the basis of the plurality of the monitoring values associated with the measured value; and
estimate a tendency of fluctuation in the measured value in the period from the tendency of fluctuation in the monitoring value on the basis of correlation data in which a correlation between the measured value and the monitoring value is predetermined.

5. The information processing apparatus according to claim 4, wherein:
the measured value is a level of blood glucose,
the monitoring value is at least one of a level of glucose included in interstitial fluid, sweat, or saliva, an electrocardiographic signal, a blood pressure, or a body temperature which has a correlation with the level of blood glucose, and
the at least one processor is configured to:
acquire timing information indicating whether the measurement time point is fasting or postprandial; and
estimate the tendency of fluctuation in the measured value in the period from the tendency of fluctuation in the monitoring value, on the basis of the correlation data corresponding to the timing indicated by timing information, in the correlation data corresponding to fasting and postprandial, respectively.

6. The information processing apparatus according to claim 4, wherein:
the measured value is a level of blood glucose,
the monitoring value is at least one of a level of glucose included in interstitial fluid, sweat, or saliva, an electrocardiographic signal, a blood pressure, or a body temperature which has a correlation with the level of blood glucose, and
the at least one processor is configured to:
acquire meal information indicating a content of a meal that a person from whom the measured value is obtained eats before the measurement of the measured value; and
estimate the tendency of fluctuation in the measured value in the period from the tendency of fluctuation in the monitoring value, on the basis of the correlation data corresponding to the content of the meal indicated by the meal information, in a plurality of pieces of the correlation data different for each content of the meal.

7. The information processing apparatus according to claim 4, wherein the at least one processor is configured to derive a range of fluctuation in the monitoring value in the period as the tendency of fluctuation in the monitoring value.

8. The information processing apparatus according to claim 4, wherein the at least one processor is configured to output a comment corresponding to the estimated tendency of fluctuation in the measured value.

9. The information processing apparatus according to claim 4, wherein the at least one processor is configured to perform first evaluation on the measured value on the basis of the estimated tendency of fluctuation in the measured value.

10. The information processing apparatus according to claim 9, wherein the at least one processor is configured to output a comment corresponding to the first evaluation.

11. The information processing apparatus according to claim 3, wherein the at least one processor is configured to:
derive a tendency of fluctuation in the monitoring value in the period on the basis of the plurality of the monitoring values associated with the measured value; and
perform first evaluation on the measured value on the basis of the tendency of fluctuation in the monitoring value.

12. The information processing apparatus according to claim 1, wherein the at least one processor is configured to perform second evaluation on the measured value on the basis of a deviation between a reference value of the monitoring value at the measurement time point indicated by the time information and the monitoring value at the measurement time point indicated by the time information.

13. The information processing apparatus according to claim 12, wherein the at least one processor is configured to output a comment corresponding to the second evaluation.

14. The information processing apparatus according to claim 1, wherein the at least one processor is configured to perform third evaluation on the measurement time point indicated by the time information on the basis of the monitoring value associated with the measured value.

15. The information processing apparatus according to claim 14, wherein the at least one processor is configured to output a comment corresponding to the third evaluation.

16. The information processing apparatus according to claim 1, wherein the at least one processor is configured to output the monitoring value associated with the measured value.

17. An information processing apparatus comprising at least one processor, wherein the at least one processor is configured to:
acquire a monitoring value which is obtained by monitoring biological information having a correlation with a measured value which is measured in a sample test; and
recommend measurement of the measured value in a case where a deviation between the monitoring value and a reference value of the monitoring value is smaller than a predetermined threshold value.

18. The information processing apparatus according to claim 1, wherein:
the measured value is a level of blood glucose, and
the monitoring value is at least one of a level of glucose included in interstitial fluid, sweat, or saliva, an electrocardiographic signal, a blood pressure, or a body temperature which has a correlation with the level of blood glucose.

19. A non-transitory computer-readable storage medium storing an information processing program for causing a computer to execute processing of:
acquiring a measured value, which is measured in a sample test, together with time information indicating a measurement time point of the measured value;
acquiring a monitoring value which is obtained by monitoring biological information having a correlation with the measured value; and
associating the measured value with the monitoring value at the measurement time point indicated by the time information.
